(19) United States
(12) Patent Application Publication
(10) Pub. No.: US 2026/0256352 A1
(43) Pub. Date: Sep. 3, 2026

(54) DETECTING BINOCULAR EYE ALIGNMENT USING SIGNAL CORRELATION

(71) Applicant: THE JOHNS HOPKINS UNIVERSITY, Baltimore, MD (US)

(72) Inventors: Boris GRAMATIKOV, Baltimore, MD (US); David Lee GUYTON, Baltimore, MD (US)

(21) Appl. No.: 18/871,568

(22) PCT Filed: Jun. 12, 2023
(86) PCT No.: PCT/US2023/025063

§ 371 Date: Dec. 4, 2024

Related U.S. Application Data
(60) Provisional application No. 63/353,058, filed on Jun. 17, 2022.

(51) Int. Cl. A61B 3/08

(52) U.S. Cl. CPC A61B 3/085

(57) ABSTRACT

Techniques for determining an alignment status of a patient's eyes are presented. The techniques can include: directing polarized light to a left retina of the patient and directing polarized light to a right retina of the patient; obtaining a left eye electrical signal corresponding to polarized light reflected from the left retina for circular scans of the left retina and obtaining a right eye electrical signal corresponding to polarized light reflected from the right retina for circular scans of the right retina; determining a correlation of a signal derived from the left eye electrical signal with a signal derived from the right eye electrical signal; determining an alignment status of the patient's eyes based on the correlation; and providing the alignment status.

### CROSS-REFERENCE TO RELATED APPLICATIONS

This application is the national stage entry of International Patent Application No. PCT/US2023/025063, filed on Jun. 12, 2023, and published as WO 2023/244537 A1 on Dec. 21, 2023, which claims the benefit of U.S. Provisional Patent Application No. 63/353,058, filed on Jun. 17, 2022, which are hereby incorporated by reference herein in their entireties.

### FIELD

This disclosure relates generally to ophthalmology.

### BACKGROUND

Retinal birefringence scanning (RBS) is an established method of detecting central fixation. With it, binocular eye alignment is declared when both eyes (Right Eye, RE, and Left Eye, LE) are fixating at the same time on a small presented target. Central fixation is assumed when the spectral power of the scanning signal returned from the retina is above a certain threshold for a characteristic frequency, or a combination of frequencies. This is typically done for each eye separately, and binocular eye alignment is declared when both eyes pass the same threshold. However, due to optical hardware asymmetries and/or the presence of certain instrumental noise (e.g., being different for the signals received for each eye), device-to-device variability, etc., applying threshold-based decision-making may result in imprecise determinations. Furthermore, pupil diameter and retinal reflectivity vary from subject to subject, causing additional uncertainties.

### SUMMARY

According to various embodiments, a system for determining an alignment status of a patient's eyes is presented. The system includes: a source of polarized light; an optical detector disposed to receive polarized light from the source upon being reflected from a left retina of the patient and produce a corresponding left eye electrical signal for circular scans of the left retina and to receive polarized light from the source upon being reflected from a right retina of the patient and produce a corresponding right eye electrical signal for circular scans of the right retina; an electronic processor communicatively coupled to the optical detector; and persistent electronic memory comprising instructions that, when executed by the electronic processor, configure the electronic processor to perform actions comprising: determining a correlation of a signal derived from the left eye electrical signal with a signal derived from the right eye electrical signal; determining an alignment status of the patient's eyes based on the correlation; and providing the alignment status.

Various optional features of the above system embodiments include the following. The alignment status may include an indication of one of: the right eye and the left eye are aligned, or the right eye and the left eye are misaligned. The signal derived from the left eye electrical signal may include a fast Fourier transform power of at least one frequency characteristic of central fixation, and the signal derived from the right eye electrical signal may include a fast Fourier transform power of at least one frequency characteristic of central fixation. The correlation may include a magnitude-squared coherence of the signal derived from the left eye electrical signal and the signal derived from the right eye electrical signal. The correlation may include a linear fit of a one of the signal derived from the left eye electrical signal or the signal derived from the right eye electrical signal to another of the signal derived from left eye electrical signal or the signal derived from the right eye electrical signal. The correlation may include a spectral correlation coefficient for a signal derived from the left eye electrical signal and a signal derived from the right eye electrical signal. The alignment status may include an identification of a misaligned eye. The actions may further include: determining a time of misalignment based on the correspondence; identifying the misaligned eye based on a comparison of the signal derived from the left eye electrical signal and the signal derived from the right eye electrical signal at the time of misalignment. The actions may further include, prior to identifying the misaligned eye: linearly fitting of one of the signal derived from left eye electrical signal or the signal derived from the right eye electrical signal to another of the signal derived from left eye electrical signal or the signal derived from the right eye electrical signal. The actions may further include: determining a time of relative alignment based on the correspondence; identifying the misaligned eye based on a comparison of the signal derived from the left eye electrical signal and the signal derived from the right eye electrical signal at the time of relative alignment.

According to various embodiments, a method of determining an alignment status of a patient's eyes is presented. The method includes: directing polarized light to a left retina of the patient and directing polarized light to a right retina of the patient; obtaining a left eye electrical signal corresponding to polarized light reflected from the left retina for circular scans of the left retina and obtaining a right eye electrical signal corresponding to polarized light reflected from the right retina for circular scans of the right retina; determining a correlation of a signal derived from the left eye electrical signal with a signal derived from the right eye electrical signal; determining an alignment status of the patient's eyes based on the correlation; and providing the alignment status.

Various optional features of the above method embodiments include the following. The alignment status may include an indication of one of: the right eye and the left eye are aligned, or the right eye and the left eye are misaligned. The signal derived from the left eye electrical signal may include a fast Fourier transform power of at least one frequency characteristic of central fixation, and the signal derived from the right eye electrical signal may include a fast Fourier transform power of at least one frequency characteristic of central fixation. The correlation may include a magnitude-squared coherence of the signal derived from the left eye electrical signal and the signal derived from the right eye electrical signal. The correlation may include a linear fit of a one of the signal derived from the left eye electrical signal or the signal derived from the right eye electrical signal to another of the signal derived from left eye electrical signal or the signal derived from the right eye electrical signal. The correlation may include a spectral correlation coefficient for a signal derived from the left eye electrical signal and a signal derived from the right eye electrical signal. The alignment status may include an identification of a misaligned eye. The method may include: determining a time of misalignment based on the correspondence; and identifying the misaligned eye based on a comparison of the signal derived from the left eye electrical signal and the signal derived from the right eye electrical signal at the time of misalignment. The method may further include, prior to the identifying the misaligned eye: linearly fitting of one of the signal derived from left eye electrical signal or the signal derived from the right eye electrical signal to another of the signal derived from left eye electrical signal or the signal derived from the right eye electrical signal. The method may further include: determining a time of relative alignment based on the correspondence; and identifying the misaligned eye based on a comparison of the signal derived from the left eye electrical signal and the signal derived from the right eye electrical signal at the time of relative alignment.

Combinations, (including multiple dependent combinations) of the above-described elements and those within the specification have been contemplated by the inventors and may be made, except where otherwise indicated or where contradictory.

### BRIEF DESCRIPTION OF THE DRAWINGS

Various features of the examples can be more fully appreciated, as the same become better understood with reference to the following detailed description of the examples when considered in connection with the accompanying figures, in which:

FIG. 1 shows a computer-generated model of the effect of human Henle fiber birefringence on the polarization state of initially linearly-polarized light at 90° reflected from the fundus, measured in terms of Stokes' S₁, along with example circular scan locations for determining central fixation;

FIG. 2 is a schematic diagram of a simplified binocular RBS system;

FIG. 3 depicts RBS signals (fast Fourier transform powers) for the left eye and right eye of a test subject, illustrating a lack of RBS signal normalization;

FIG. 4 schematically depicts obtaining RBS signals during central fixation with good alignment between a patent's right eye and left eye, according to various embodiments;

FIG. 5 schematically depicts obtaining RBS signals during para-central fixation with good alignment between a patient's right eye and left eye, according to various embodiments;

FIG. 6 schematically depicts obtaining RBS signals during eye misalignment, where a patient's left eye is off-center, according to various embodiments;

FIG. 7 shows a chart depicting the magnitude-squared coherence (MSC) for central fixation frequencies and a chart depicting MSC for off-central fixation frequencies for a test subject, according to various embodiments;

FIG. 8 depicts the phase of the cross-spectral density for the data depicted in FIG. 7, according to various embodiments;

FIG. 9 depicts the characteristic powers for a 1f2f system for a test subject, plotted over time, according to various embodiments;

FIG. 10 illustrates the performance of a linear fit for a spHWP system, according to various embodiments;

FIG. 11 illustrates logic of an algorithm for identifying a misaligned eye, according to various embodiments; and

FIG. 12 is a flow diagram for a method of determining an alignment status of a patient's eyes, according to various embodiments.

### DESCRIPTION OF THE EXAMPLES

Reference will now be made in detail to example implementations, illustrated in the accompanying drawings. Wherever convenient, the same reference numbers will be used throughout the drawings to refer to the same or like parts. In the following description, reference is made to the accompanying drawings that form a part thereof, and in which is shown by way of illustration specific exemplary examples in which the invention may be practiced. These examples are described in sufficient detail to enable those skilled in the art to practice the invention and it is to be understood that other examples may be utilized and that changes may be made without departing from the scope of the invention. The following description is, therefore, merely exemplary.

### I. Introduction

Some embodiments presented here avoid absolute measurements of spectral power, while focusing on the spectral similarities (or lack thereof), in order to establish a reliable determination of binocular eye alignment. Some embodiments utilize correlation between signals from each eye to determine an alignment status of a patient's eyes, without requiring normalization. These and other features and advantages are presented herein in reference to the figures.

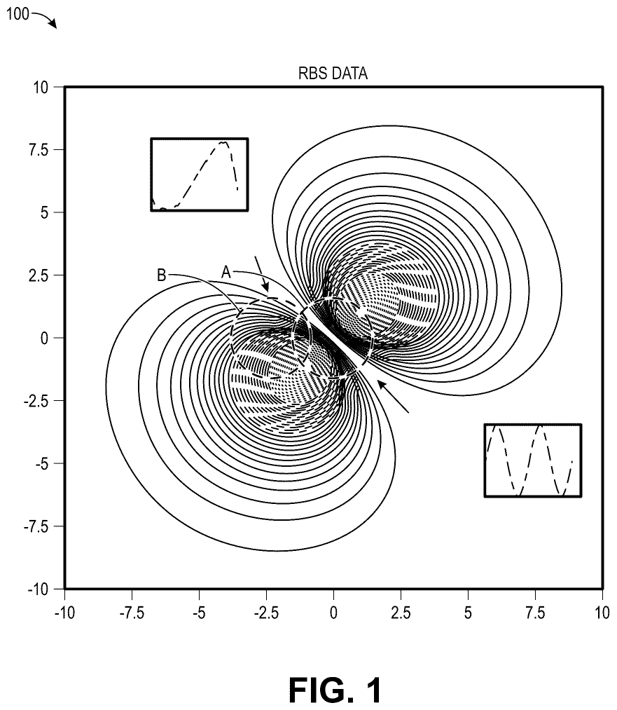

In general, there are two main types of RBS systems. The first type, referred to herein as a “1f2f” system, uses simple circular scans around the presumed location of the fovea. For a 1f2f system, polarized, e.g., near-infrared, light is reflected from the foveal area in a detectable bow-tie-like pattern of polarization states (see FIG. 1), allowing localization and eye tracking. The fovea is aimed at the object of regard during fixation. When illuminated with polarized (e.g., linearly polarized, circularly polarized, elliptically polarized, etc.) near-infrared light, such as the light emitted by a low-power 785 nm or 830 nm laser diode, the uniquely arranged, radially symmetric nerve fibers (Henle fibers) surrounding the center of the fovea change the polarization state of the light being back-reflected from the underlying retinal pigment epithelium.

FIG. 1 shows a computer-generated model 100 of the effect of human Henle fiber birefringence on the polarization state of initially linearly-polarized light at 90° reflected from the fundus, measured in terms of Stokes' S₁, along with example circular scan locations for determining central fixation. The Henle fibers are the uniquely-arranged, radially-symmetric nerve fibers surrounding the fovea. The axes are in degrees measured from the foveal center. Circular scan (counterclockwise) locations are indicated by dotted circles, and start at the three o'clock position. As described presently, such circular scans can help establish central fixation (a), or lack thereof (b). The time signals are for a 1f2f system.

Thus, FIG. 1 shows the distribution (Haidinger brush) of Stokes component S₁ (horizontal preference) around the center of the fovea, as a combination of a fundus image taken using polarized light and a polarizer, and a superimposed graphical printout from a computer model. The location of this “brush” can be detected by interrogating the area with a full raster scan (which is possible, but would be relatively slow), with just a few laser beam spots, or by means of a fast circular scan.

To detect central fixation, e.g., in the case of pediatric vision screeners, circular scanning followed by frequency analysis may be used. When the eye fixates on a fixation target optically at the center of the scanning circle, the returned scan signal s(t) is of a specific frequency f₂, such as twice the scanning frequency fₛ, as represented by scan (A) in FIG. 1. Alternatively, off-center fixation produces a different signal, of merely the scanning frequency for example, f₁=fₛ, as represented by scan (B) in FIG. 1. In both cases, for FIG. 1, the scanning starts at an imaginary three o'clock position on the scanning circle, shown with dotted lines, and progresses in a counter-clockwise direction. Off-center directions of gaze produce mixtures of the two frequencies and signal traces specific to the direction of gaze.

In the above described circular RBS method, the signal level is very low, because the returned light from the retina is approximately 5000 times less than the light of the scanning beam entering the eye, and is comparable with the instrumental noise. Prior art techniques have attempted to handle this problem using background subtraction (flat fielding), which slows down system performance. As an alternative, in more recent, second type, RBS systems, spatial polarization modulation was introduced, incorporating a double-pass half wave plate (HWP) spinning 9/16th as fast as the circular scan frequency fₛ. The spinning HWP works as a polarization rotator. When interacting with the Henle fibers, the rotating polarization of the incident light modulates the RBS signal and generates half-multiples of the scanning frequency upon reflection. The characteristic frequencies for this system are 2.5fₛ and 6.5fₛ for central fixation, and 3.5fₛ and 5.5fₛ for off-central fixation. These half-multiple frequency signals double in amplitude and even quadruple in signal strength (fast Fourier transform power) with 360° phase-shift subtraction, whereas most of the optical background noise (instrumental noise) at whole multiples of the scanning frequency is removed, thus eliminating the need of background subtraction, and significantly increasing the signal-to-noise ratio (SNR). This second type of RBS design is referred to herein as an spHWP system.

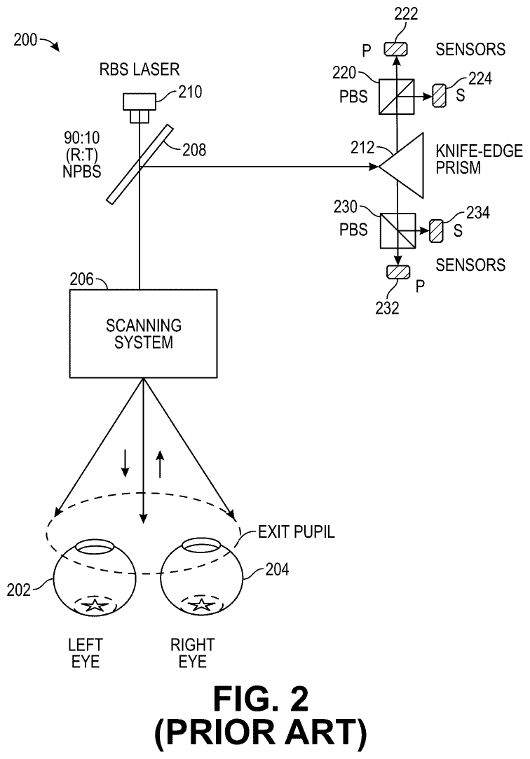

FIG. 2 is a schematic diagram of a simplified generic binocular RBS system 200. An RBS laser 210, e.g., a laser diode, provides polarized near-infrared light to an RBS scanning system 206 through a 90:10 non-polarizing beam splitter (NPBS) 208. The RBS scanning system 206 scans the light in a circular pattern with a frequency of fₛ, and directs it to the subject's left eye 202 and right eye 204. For the a spHWP RBS system, the RBS scanning system 206 incorporates a spinning HWP. A central fixation target (depicted using stars on the retinas of eyes 202, 204) optically conjugate to the plane of the scanning circle is introduced, e.g., by a beam splitter. The light reflected from the retinas of the left eye 202 and right eye 204 is redirected by the NPBS 208 to a knife-edge reflecting prism 212, which then separates the light coming from the two eyes 202, 204. For each eye 202, 204, a polarizing beamsplitter (PBS) 220, 230 decomposes, i.e., splits, the light into s- and p-components, which after measurement by corresponding sensors 222, 224, 232, 234 are used to build the Stokes vector component S₁: S₁=s-p. Not shown are waveplates, mirrors, lenses, light traps, polarization rotators, polarization compensators, scanning motor, scanning mirrors, etc.

In both types of RBS systems, central fixation is determined when the spectral power of the scanning signal returned from the retina is above a certain threshold for a characteristic frequency or combination of frequencies. This is usually done for each eye separately, and central fixation is determined when both eyes pass the same threshold. However, due to optical hardware asymmetries and/or the presence of certain instrumental noise (different for the signals received for the two eyes), device-to-device variability etc., applying a threshold-based decision-making may become imprecise and produce erroneous results. Furthermore, pupil diameter and retinal reflectivity vary from subject to subject. Finally, the position of the eye in the exit pupil of the device can also affect the signal amplitude. Some embodiments address all of the above sources of variability and asymmetry through the use of correlation of signals from both eyes.

### II. Illustration of the Problem

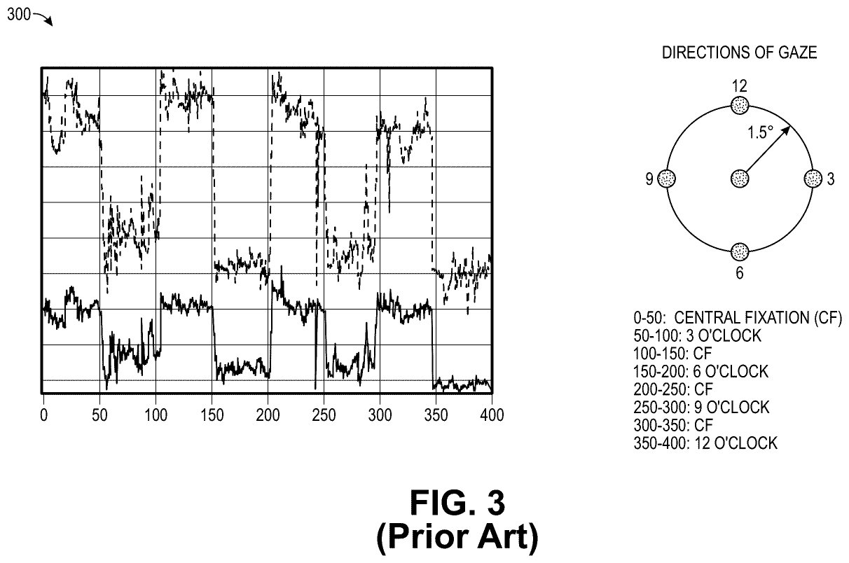

FIG. 3 depicts RBS signals (fast Fourier transform powers) 300 for the left eye and right eye of a test subject, illustrating the problem of using a single threshold for both eyes. The RBS signals represented by chart 300 of FIG. 3 are for the second Stokes vector component S₁, or s-p. Spectral powers at central-fixation-characteristic frequencies over time are depicted, while the test subject was fixating on a central target during acquisitions 0-50, 100-150, 200-250, and 300-350. At all other times the subject was fixating on points 1.5° away from the central target (off-central fixation). The traces are unfiltered and are from an spHWP system. The top trace represents the subject's right eye, and the bottom trace represents the subject's left eye. Each trace represents the combined power P25+P65 (for central-fixation-characteristic frequencies 2.5fₛ and 6.5fₛ, respectively), over a period of ~80 s (400 acquisitions). The spectra were obtained from component S₁ of the Stokes vector. Each data point was derived from the fast Fourier transform obtained from a time-domain RBS signal acquisition of duration 200 ms.

As is apparent from FIG. 3, while the test subject's vision was normal and he was responding adequately to instructions to change the direction of gaze, the traces are different, most likely because the two channels have different gain and bias, or the two eyes are in a different position in the exit pupil of the device. Some pupil size asymmetry, or even cataract, could also have been the cause. This would prevent the software designer from using simple thresholds applied naïvely to the power spectrum. For example, no single threshold separates central fixation from non-central fixation for both eyes.

### III. Indicators of Similarity

The magnitude-squared coherence (MSC) between two time-domain signals x(t) and y(t) is a real-valued function that may be defined as follows, by way of non-limiting example:

$$\\ \begin{matrix}{MSC}_{xy} ( f ) = \frac{{\vert {G}_{xy} ( f ) \vert}^{2}}{{G}_{xx} ( f ) {G}_{yy} ( f )} & ( 1 )\end{matrix}$$

In Equation (1), G_{xy}(f) represents the cross-spectral density between x and y, and Gₓₓ(f) and G_{yy}(f) represent the autospectral densities of x and y, respectively. The magnitude of the cross-spectral density is denoted as |G|. The MSC is a measure of similarity in the frequency content of two signals. According to various embodiments, the two signals are frequency powers for the right eye and the left eye, respectively. The MSC_{xy}(f) is in the range [0 . . . 1]. For ideal spectral linkage between the two signals x(t) and y(t), the coherence will be equal to one.

The cross-spectral density in the above formula is calculated based on the single-sided, scaled cross-power spectrum of the two discrete time-domain signals, which may be expressed as follows, by way of non-limiting example:

$$\\ \begin{matrix}{G}_{xy} = \frac{FFT ( x ) FFT ( y )}{{n}^{2}} & ( 2 )\end{matrix}$$

In Equation (2), n represents the number of sample points, and x and y are the time-domain RBS signals from the right eye (RE) and the left eye (LE), respectively. According to some embodiments, the fast Fourier transform is calculated from time epochs of between 100 ms and 1 s inclusive, depending on the requirements for speed vs. frequency resolution. Time epochs may be overlapping or non-overlapping.

Because the fast Fourier transform is complex-valued, so is G_{xy}, and therefore it can be considered comprised of a magnitude magG_{xy}, also |G_{xy}| in Equation (1) above, and phase phaseG_{xy}. The single-sided phase shows the difference between the phases of signals x and y, the right-eye and left-eye signals, and is indicative of difference in the direction of gaze.

The Correlation Coefficient (CC) as a general measure of similarity can be calculated using the basic formula for the Pearson correlation coefficient for a sample may be expressed as follows, by way of non-limiting example:

$$\\ \begin{matrix}r = \frac{\sum ( {x}_{i} - \overline{x} ) ( {y}_{i} - \overline{y} )}{\sqrt{\sum {( {x}_{i} - \overline{x} )}^{2} \sum {( {y}_{i} - \overline{y} )}^{2}}} & ( 3 )\end{matrix}$$

In Equation (3), x̅ and y̅ represent the mean values of the respective variables.

### IV. Determining Eye Alignment Status

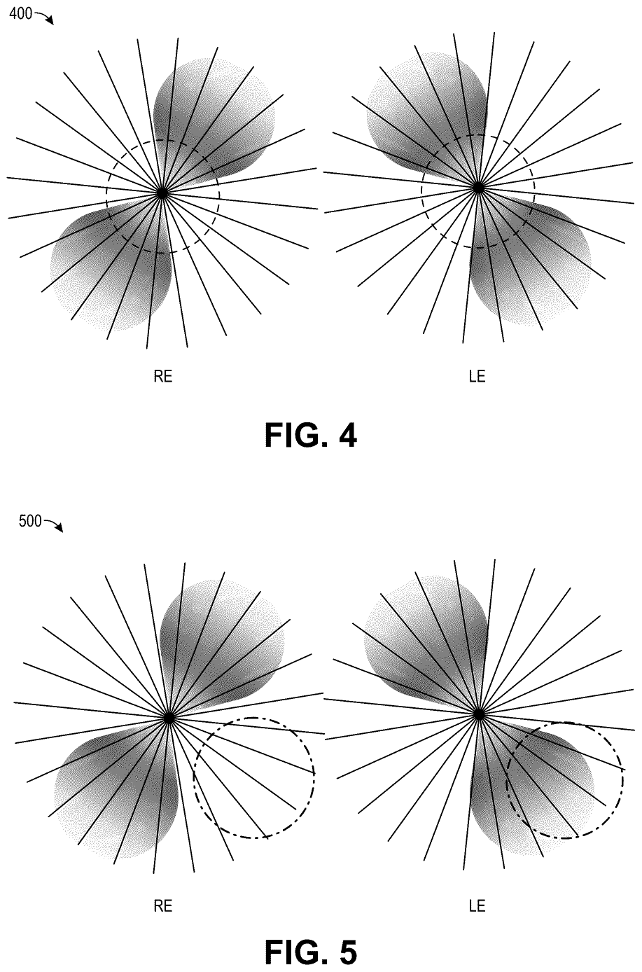

FIG. 4 schematically depicts obtaining RBS signals 400 during central fixation with good alignment between a patient's right eye and left eye, according to various embodiments. In particular, FIG. 4 shows circular scanning around the centers of two patient foveas during central fixation and with good binocular eye alignment. Consider the application of the MSC, e.g., Equation (1) to the signals acquired by a 1f2f system. During central fixation, the dominant spectral powers are 2fₛ for both eyes. Because for MSC the phase of the scan does not matter, in the ideal case the MSC is expected to be close to 1, regardless of the time domain signal amplitudes from the two eyes. For a spHWP system, the situation is similar, where the MSC is calculated for 2.5fₛ and 6.5fₛ.

FIG. 5 schematically depicts obtaining RBS signals 500 during para-central fixation with good alignment between a patient's right eye and left eye, according to various embodiments. Both foveas are shifted by an equal amount in the same direction. Even though the eyes may be well aligned, the scanning circle does not encompass the centers of the two foveas. The scan paths go along very different areas of the two foveas. Consequently, neither spectral coherence nor phase similarity is to be expected, and the MSC will stay low.

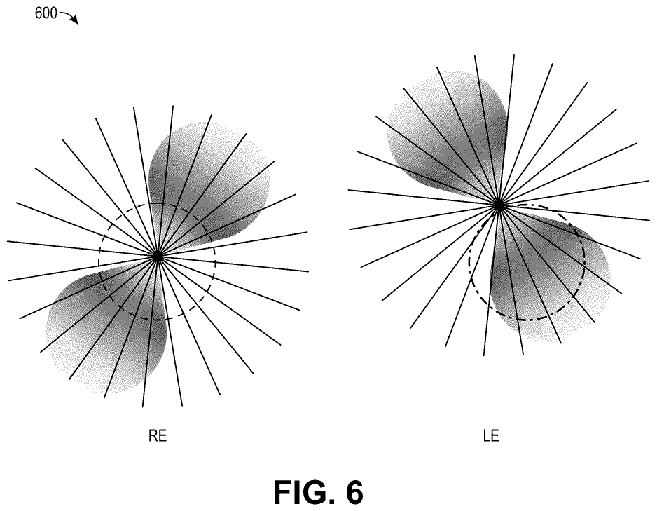

FIG. 6 schematically depicts obtaining RBS signals 600 during eye misalignment, where a patient's left eye is off-center, according to various embodiments. In this case, the MSC will stay low, where the RBS signals represent true misalignment of the patient's eyes. While the right eye is aligned with the presented target and the scan goes around the center of the right eye fovea, the fovea of the left eye is not in the center of the scanning circle, and therefore this misalignment will generate a different dominant frequency. For this case, a 1f2f system will produce a strong 2fₛ spectral component for the right eye, while the left eye will give rise to a predominantly 1fₛ component, which may immediately be detected by the MSC based solely on spectral content. Similarly, with a spHWP system, the right eye may generate 2.5fₛ and 6.5fₛ spectral powers, while the left eye may produce 3.5fₛ and 5.5fₛ spectral powers.

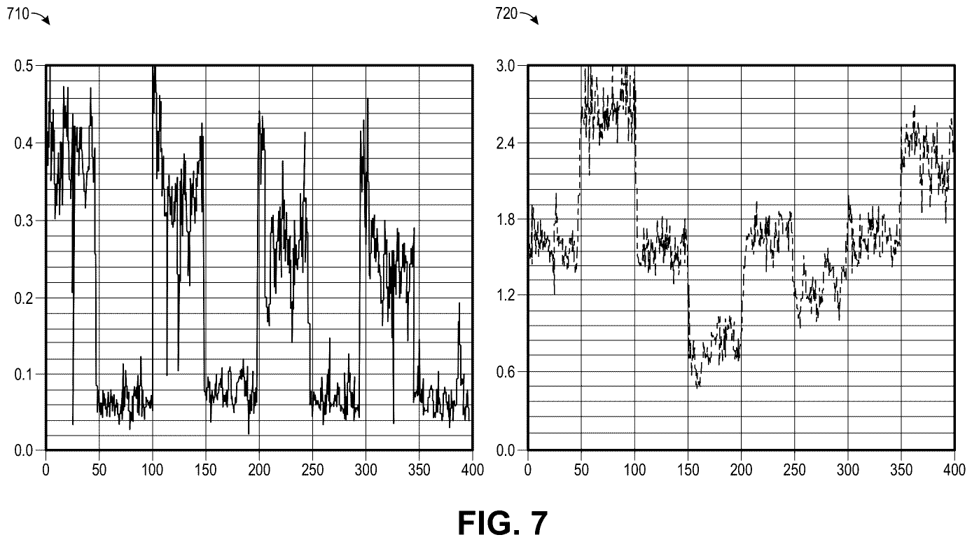

FIG. 7 shows a chart 710 depicting MSC for central fixation frequencies and a chart 720 depicting MSC for off-central fixation frequencies for a test subject, according to various embodiments. In particular, the charts 710, 720 depict MSC (from a spHWP system) for a test subject who was asked to look at a presented central target during acquisitions 0-50, 100-150, 200-250, and 300-350, and for the remaining times, was asked to look 1.5° away in different directions (at 3, 6, 9 and 12 o'clock on the rim of the 3° scanning circle encircling the central target). The chart 710 depicts MSC for the central fixation frequencies P2565, calculated for 2.5fₛ and 6.5fₛ. As seen in the chart 710, the MSC is high during central fixation and low during off-central fixation. As is apparent from the chart 710, a clear threshold (about 0.15) separates central fixation from off-central fixation. This distinction may be used by various embodiments. The chart 720 depicts MSC for the off-central fixation frequencies P3555, calculated for 3.5fₛ and 5.5fₛ. As seen in the chart 720, the MSC stays at a certain constant level during central fixation (between about 1.3 and 1.8), but is either above or below this level during off-central fixation. For the charts 710, 720, the fast Fourier transform traces were not filtered.

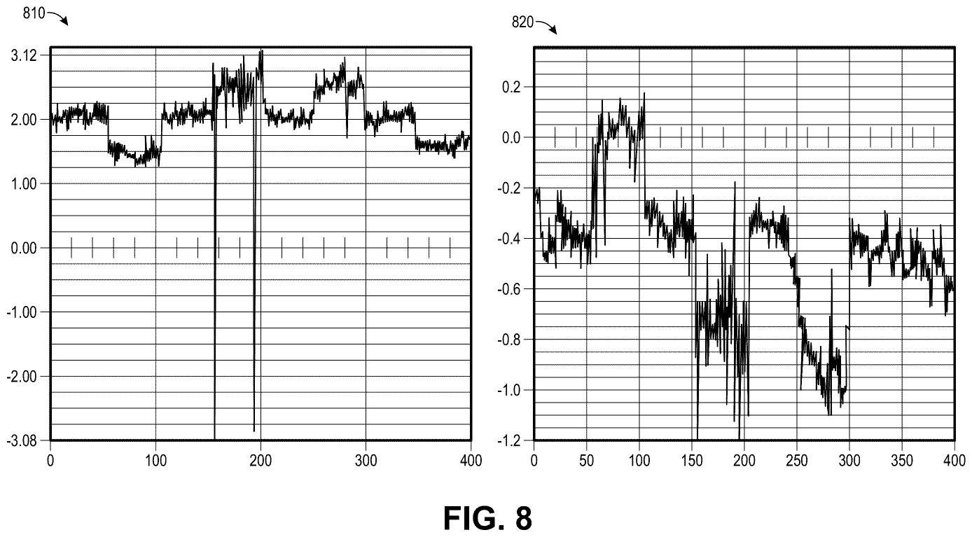

In addition, because of a significant difference between the phases, the phaseG_{xy}, derived from the complex-valued Gxy, may also be used to detect the spectral discrepancy and signal misalignment. The off-center spectral powers are more responsive to off-center fixation than the central fixation powers. This is illustrated by FIG. 8.

FIG. 8 depicts the phase phaseG_{xy} of the cross-spectral density G_{xy} for the data depicted in FIG. 7, according to various embodiments. The chart 810 corresponds to the chart 710, and the chart 820 corresponds to the chart 720. The chart 810 depicts Sxy=phaseG_{xy} for 3.5fₛ, and the chart 820 depicts Sxy=phaseG_{xy} for 5.5fₛ. In FIG. 8, the phaseG_{xy} responds to every deviation from central fixation. As illustrated by the charts 810, 820, both frequency components move away from their baseline values (here 2.0 for 3.5fₛ and −0.4 for 5.5fₛ).

Thus, similar to the magnitude MSC, the phase tends to stay constant during central fixation, and deviates from this level during off-central fixation. This can be utilized according to various embodiments to identify moments of central fixation, or lack thereof.

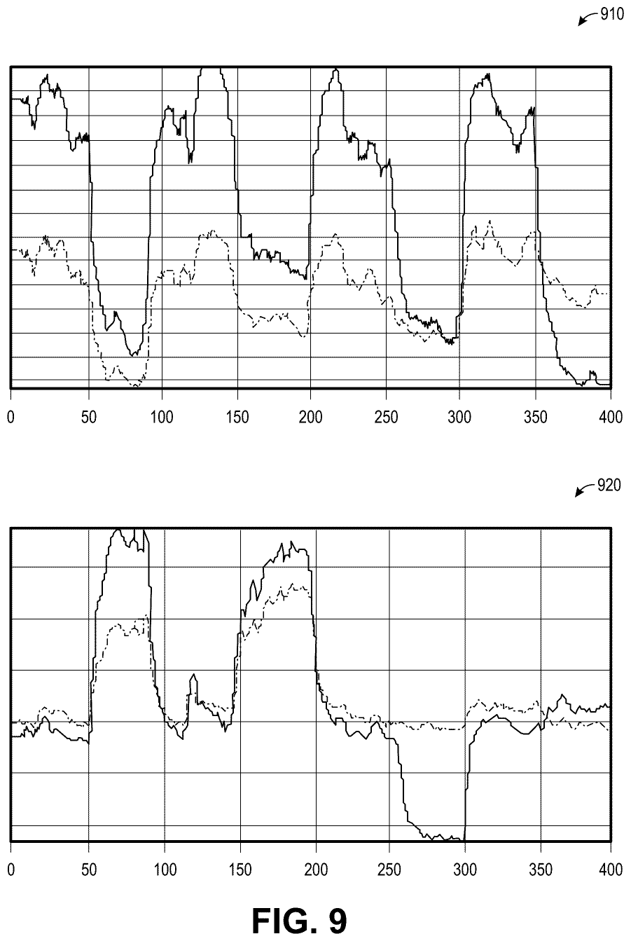

A Spectral Correlation Coefficient (SCC) used in the present context treats the signals x and y as the spectral powers (for right eye and left eye, respectively) for a certain frequency (or a combination of central-fixation-characteristic frequencies), followed over a sufficiently long period of time. Thus, for a 1f2f system, an embodiment may use the fast Fourier transform powers for f=2fₛ as a function of time, e.g., P2^{RE}(t) for the right eye and P2^{LE}(t) for the left eye. An example for a 1f2f system is shown in FIG. 9.

FIG. 9 depicts the characteristic powers for a 1f2f system for a test subject, plotted over time, according to various embodiments. The test subject was asked to look at a presented central target during acquisitions 0-50, 100-150, 200-250, and 300-350. The traces are filtered with a low-pass filter. The chart 910 depicts the powers at 2fₛ (P2) for the right eye (dashed) and the left eye (solid). P2 is high during central fixation, and lower during off-central fixation. The Spectral Correlation Coefficient is SCC=0.8159 (based on P2 only). The chart 920 depicts the powers at 1fₛ (P1) for the right eye (dashed) and left eye (solid). P1 is typically at its baseline during central fixation, and higher or lower during off-central fixation.

As is apparent from FIG. 9, the central fixation power traces (chart 910) for the two eyes are quite similar in shape, in spite of differences in amplitude and bias. This is where the SCC may be used according to various embodiments. From a signal processing point of view, high SCC calculated from the central fixation traces indicates good spectral similarity, hence good binocular eye alignment, while a lower SCC is a sign of spectral dissociation, and most likely binocular eye misalignment. By way of non-limiting example, a reasonable threshold for binocular eye alignment may be around SCC=0.8.

The correlation approach may be used together with an equalizing function according to some embodiments. This is practical, for example, when the two channels (right eye versus left eye) have different offset and gain, due to hardware asymmetries, or asymmetry between the eyes (e.g., pupil size, optical clarity, refractive error, retinal reflectivity, etc.). In this case, an attempt can be made to make the signal in the weaker (or biased) channel as equal as possible to the other, stronger channel. This may be done by calculating two parameters, slope (a) and intercept (b), such that each value in the equalized channel yᵢ can be represented as closely as possible by zᵢ, such that the i-th element of the balanced array yᵢ becomes zᵢ, e.g., as represented by non-limiting example as follows:

$$\\ \begin{matrix}{z}_{i} = {ax}_{i} + b & ( 4 )\end{matrix}$$

In Equation (4), the slope (a) and the intercept (b) values represent best the linear fit of the data points x (for the right eye) and y (for the left eye) and may be calculated using the least squares method. A measure of the goodness of the fit is the mean squared error, mse, which may be represented by non-limiting example as follows:

$$\\ \begin{matrix}mse = \frac{{\sum }_{i = 0}^{n - 1} {\vert {x}_{i} - {y}_{i} \vert}^{2}}{n} & ( 5 )\end{matrix}$$

Once the linear fit operation is performed in order to balance the channels to the maximum, the spectral correlation coefficient SCC may be calculated for the time period where central fixation was attempted by the test subject. Again, high SCC indicates good spectral similarity, hence good binocular eye alignment, while a lower SCC is a sign of spectral dissociation, and most likely binocular eye misalignment. A high value for the mse by itself is an indicator of failing fit and poor binocular eye alignment.

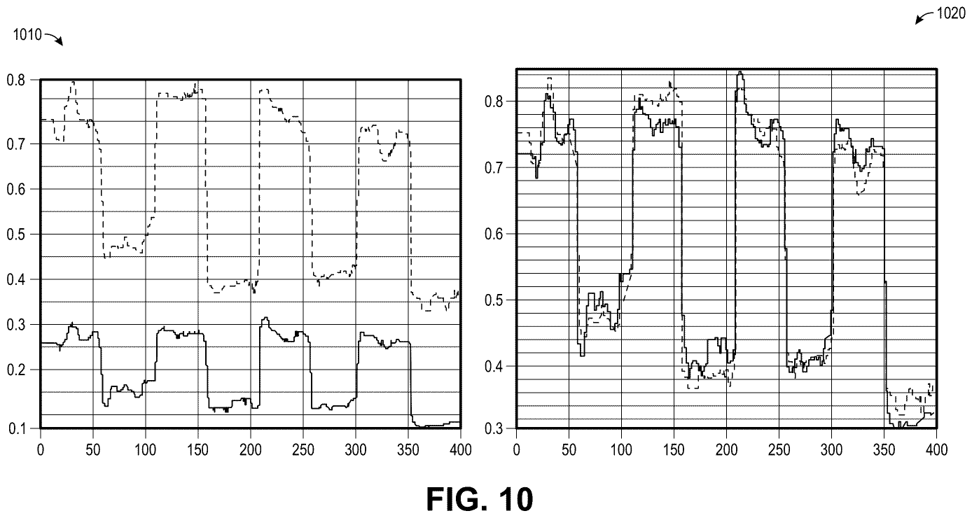

FIG. 10 illustrates the performance of a linear fit for a spHWP system, according to various embodiments. The horizontal axis is the time measured in terms of acquired and analyzed epochs. The chart 1010 shows the traces for the right eye (dashed) and left eye (solid), each representing the normalized central fixation power P2565/P45. (P2565 is the sum of the fast Fourier transform powers for 2.5fₛ and 6.5fₛ.) The power at 4.5fₛ may be stable enough to use for normalization. However, the traces are disbalanced due to hardware dissimilarities in the two channels.

The chart 1020 shows the same traces as are depicted in the chart 1010 after re-balancing using the linear fit according to Equation (4). The re-balancing has increased the signal levels for the left eye. The re-balanced left eye channel is shown solid; the unchanged right eye trace is dashed. The resulting slope is 2.23, while the intercept is 0.145. The mse is 0.0009, indicating an excellent fit, which together with a final SCC of 0.98592 means excellent binocular eye alignment. According to some embodiments, and as shown in FIG. 10, a threshold of 0.6 may be used to separate central fixation from para-central fixation for both channels after rebalancing.

The performance of the linear fit for a 1f2f system is similar.

In general, some embodiments may identify which eye is misaligned. For example, after establishing misalignment by comparing MSC with a threshold value, some embodiments identify which of the two eyes is misaligned. This can be achieved following the algorithm presented below, by way of non-limiting example:

(1) Calculate the MSC for the central fixation frequencies.

(2) Identify a moment of misalignment, e.g., a low MSC (e.g., as shown and described in reference to the chart 710 of FIG. 7). Save the time location of the misalignment (t_{M}).

(3) Do a channel re-balancing for the central fixation frequencies (e.g., as shown and described in reference to chart 1020 of FIG. 10). For the spHWP system, re-balancing may be done on the P2565/P45 traces. For 1f2f systems, re-balancing may be done on the P2 traces.

(4) In the re-balanced array, go to the time location of the misalignment (t_{M}) and compare the powers (P2565 for a spHWP system, or P2 for a 1f2f system). The eye with the lower power is the eye looking off-center.

This algorithm may be applied for several time moments of misalignment. The following optional additional steps may improve precision of the algorithm.

(5) Define a moment of relative alignment (if any), typically a high MSC (e.g., as shown and described in reference to the chart 710 of FIG. 7). Save the time location of the relative alignment (t_{A}).

(6) In the re-balanced central fixation array, go to the time location of the maximum alignment (tₐ), if any, and compare the powers (e.g., P2565 for a spHWP system, or P2 for a 1f2f system). The eye with significantly lower central fixation power is the eye looking off-center.

The algorithm may be implemented in a system using software according to various embodiments. It has been shown to function for 1f2f systems, as well as for spHWP systems. It does not depend on the absolute powers (P2 or P2565).

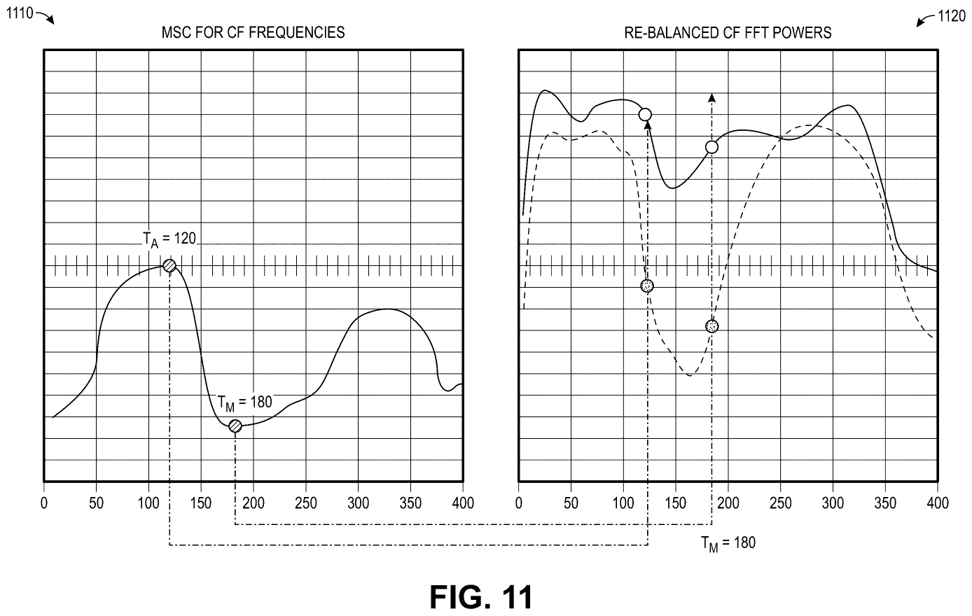

FIG. 11 depicts charts 1110, 1120 illustrating the logic of the algorithm for identifying a misaligned eye, according to various embodiments. The chart 1110 represents the MSC for the central fixation frequencies, while the chart 1120 shows the re-balanced central fixation powers of the fast Fourier transform for the two eyes (dashed for the right eye and solid for the left eye). The central fixation frequency for a 1f2f system may be 2fₛ, whereas for a spHWP system this may be 2.5fₛ and 6.5fₛ. The horizontal axes stand for the time in terms of epochs of acquired and analyzed RBS signals. First, the MSC for the central fixation frequencies is calculated, and the time t_{M} of its minimum is registered (here t_{M}=180). This is the time moment of the least spectral similarity and is understood to be a moment of misalignment. Making a judgement as to which is the misaligned eye uses the chart 1120, where the re-balanced central fixation powers are. At t_{M}, the spectral power for central fixation is much higher for the left eye (solid), therefore logically we can assume that the right eye is the misaligned eye (not fixating on the presented target). In a similar manner, the point t_{A} of the maximum MSC for central fixation may be used (here t_{A}=120). This does not have to indicate very good alignment, but at least can serve as a moment of maximum spectral similarity. Also here, it is expected that the misaligned eye will have a lower central fixation spectral signature.

Some RBS systems use background subtraction to reduce instrumental noise, e.g., as caused by internal reflections. However, background subtraction requires non-trivial processing time, and can be uncertain during transient processes, such as blinking, etc. Unlike these approaches, identifying binocular eye alignment using correlation such as the MSC for the central fixation frequencies is fundamentally noise immune, because it operates only on the frequencies characteristic for central fixation. In the 1f2f systems this is 2fₛ, while in the spHWP systems these are the 2.5fₛ and 6.5fₛ. Non-central fixation frequencies may not be used; therefore the instrumental noise cannot interfere with the system's precision.

Some embodiments may utilize low-pass filtering of the central fixation powers to achieve more stable operation. As can be seen from FIG. 2, these traces can be quite noisy, which gives rise to noise to the trace for the MSC (see FIG. 7) and its phase (see FIG. 8). The noise is due to natural instability of the eye's fixation and is inevitable. This is why low-pass filtering of the trace for the central fixation powers can be beneficial and stabilizing. According to some embodiments, a low-pass third order Butterworth filter can remove the jitter almost entirely, as evident from FIGS. 9 and 10.

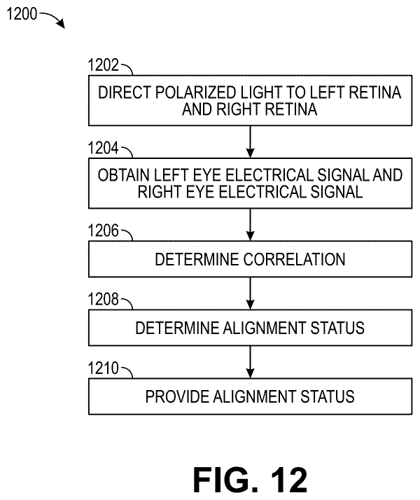

FIG. 12 is a flow diagram for a method 1200 of determining an alignment status of a patient's eyes, according to various embodiments. The method 1200 may be used with an RBS system, such as is shown and described herein in reference to FIG. 2.

At 1202, the method 1200 includes directing polarized light to a left retina of the patient and directing polarized light to a right retina of the patient. The polarized light may be directed as shown and described herein in reference to FIG. 2, for example.

At 1204, the method 1200 includes obtaining a left eye electrical signal (e.g., fast Fourier transform power) corresponding to polarized light reflected from the left retina for circular scans of the left retina and obtaining a right eye electrical signal (e.g., fast Fourier transform power) corresponding to polarized light reflected from the right retina for circular scans of the right retina. The electrical signals may be obtained as shown and described herein in reference to FIG. 2, for example.

At 1206, the method 1200 includes determining a correlation of a signal derived from the left eye electrical signal with a signal derived from the right eye electrical signal. The signal derived from the left eye electrical signal may include a fast Fourier transform power of at least one frequency characteristic of central fixation, and the signal derived from the right eye electrical signal may include a fast Fourier transform power of at least one frequency characteristic of central fixation, e.g., as shown and described herein in reference to FIG. 9.

Any of a variety of correlations disclosed herein may be used. According to some embodiments, the correlation may include use of a magnitude-squared coherence of the signal derived from the left eye electrical signal and the signal derived from the right eye electrical signal, e.g., as shown and described in reference to FIG. 7. According to some embodiments, the correlation may include use of a phase of a cross-spectral density for the signal derived from the left eye electrical signal and the signal derived from the right eye electrical signal, e.g., as shown and described in reference to FIG. 8. According to some embodiments, the correlation may include use of a spectral correlation coefficient for a signal derived from the left eye electrical signal and a signal derived from the right eye electrical signal, e.g., as shown and described in reference to FIG. 9. According to some embodiments, the correlation may include use of a linear fit of, for example, the signal derived from the left eye electrical signal to the signal derived from right eye electrical signal (or vice versa), e.g., as shown and described in reference to FIG. 10.

At 1208, the method 1200 includes determining an alignment status of the patient's eyes based on the correlation. The alignment status may include an indication of one of: the right eye and the left eye are aligned, or the right eye and the left eye are misaligned. The alignment status may include an identification of a misaligned eye.

At 1210, the method 1200 includes providing the alignment status. The alignment status may be provided by displaying it on a monitor, for example. Alternately, or in addition, the alignment status may be provided to a different system, such as a health care database system, for example.

Certain examples can be performed using a computer program or set of programs. The computer programs can exist in a variety of forms both active and inactive. For example, the computer programs can exist as software program(s) comprised of program instructions in source code, object code, executable code or other formats; firmware program(s), or hardware description language (HDL) files. Any of the above can be embodied on a transitory or non-transitory computer readable medium, which include storage devices and signals, in compressed or uncompressed form. Exemplary computer readable storage devices include conventional computer system RAM (random access memory), ROM (read-only memory), EPROM (erasable, programmable ROM), EEPROM (electrically erasable, programmable ROM), flash memory, and magnetic or optical disks or tapes.

Aspects of the present disclosure are described herein with reference to flowchart illustrations and/or block diagrams of methods, apparatus (systems), and computer program products according to embodiments of the disclosure. It will be understood that each block of the flowchart illustrations and/or block diagrams, and combinations of blocks in the flowchart illustrations and/or block diagrams, can be implemented using computer readable program instructions that are executed by an electronic processor.

These computer readable program instructions may be provided to a processor of a general-purpose computer, special purpose computer, or other programmable data processing apparatus to produce a machine, such that the instructions, which execute via the electronic processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

In embodiments, the computer readable program instructions may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the C programming language or similar programming languages. The computer readable program instructions may execute entirely on a user's computer, partly on the user's computer, as a stand-alone software package, partly on the user's computer and partly on a remote computer or entirely on the remote computer or server.

As used herein, the terms “A or B” and “A and/or B” are intended to encompass A, B, or {A and B}. Further, the terms “A, B, or C” and “A, B, and/or C” are intended to encompass single items, pairs of items, or all items, that is, all of: A, B, C, {A and B}, {A and C}, {B and C}, and {A and B and C}. The term “or” as used herein means “and/or.”

As used herein, language such as “at least one of X, Y, and Z,” “at least one of X, Y, or Z,” “at least one or more of X, Y, and Z,” “at least one or more of X, Y, or Z,” “at least one or more of X, Y, and/or Z,” or “at least one of X, Y, and/or Z,” is intended to be inclusive of both a single item (e.g., just X, or just Y, or just Z) and multiple items (e.g., {X and Y}, {X and Z}, {Y and Z}, or {X, Y, and Z}). The phrase “at least one of” and similar phrases are not intended to convey a requirement that each possible item must be present, although each possible item may be present.

The techniques presented and claimed herein are referenced and applied to material objects and concrete examples of a practical nature that demonstrably improve the present technical field and, as such, are not abstract, intangible or purely theoretical. Further, if any claims appended to the end of this specification contain one or more elements designated as “means for [perform]ing [a function] . . . ” or “step for [perform]ing [a function] . . . ”, it is intended that such elements are to be interpreted under 35 U.S.C. § 112(f). However, for any claims containing elements designated in any other manner, it is intended that such elements are not to be interpreted under 35 U.S.C. § 112(f).

While the invention has been described with reference to the exemplary examples thereof, those skilled in the art will be able to make various modifications to the described examples without departing from the true spirit and scope. The terms and descriptions used herein are set forth by way of illustration only and are not meant as limitations. In particular, although the method has been described by examples, the steps of the method can be performed in a different order than illustrated or simultaneously. Those skilled in the art will recognize that these and other variations are possible within the spirit and scope as defined in the following claims and their equivalents.

What is claimed is:

1. A system for determining an alignment status of a patient's eyes, the system comprising:
a source of polarized light;
an optical detector disposed to receive polarized light from the source upon being reflected from a left retina of the patient and produce a corresponding left eye electrical signal for circular scans of the left retina and to receive polarized light from the source upon being reflected from a right retina of the patient and produce a corresponding right eye electrical signal for circular scans of the right retina;
an electronic processor communicatively coupled to the optical detector; and
persistent electronic memory comprising instructions that, when executed by the electronic processor, configure the electronic processor to perform actions comprising:
determining a correlation of a signal derived from the left eye electrical signal with a signal derived from the right eye electrical signal;
determining an alignment status of the patient's eyes based on the correlation; and
providing the alignment status.

2. The system of claim 1, wherein the alignment status comprises an indication of one of: the right eye and the left eye are aligned, or the right eye and the left eye are misaligned.

3. The system of claim 1, wherein the signal derived from the left eye electrical signal comprises a fast Fourier transform power of at least one frequency characteristic of central fixation, and wherein the signal derived from the right eye electrical signal comprises a fast Fourier transform power of at least one frequency characteristic of central fixation.

4. The system of claim 1, wherein the correlation comprises a magnitude-squared coherence of the signal derived from the left eye electrical signal and the signal derived from the right eye electrical signal.

5. The system of claim 1, wherein the correlation comprises a linear fit of a one of the signal derived from the left eye electrical signal or the signal derived from the right eye electrical signal to another of the signal derived from left eye electrical signal or the signal derived from the right eye electrical signal.

6. The system of claim 1, wherein the correlation comprises a spectral correlation coefficient for a signal derived from the left eye electrical signal and a signal derived from the right eye electrical signal.

7. The system of claim 1, wherein the alignment status comprises an identification of a misaligned eye.

8. The system of claim 7, wherein the actions further comprise:
determining a time of misalignment based on the correspondence; and
identifying the misaligned eye based on a comparison of the signal derived from the left eye electrical signal and the signal derived from the right eye electrical signal at the time of misalignment.

9. The system of claim 8, wherein the actions further comprise, prior to identifying the misaligned eye:
linearly fitting of one of the signal derived from left eye electrical signal or the signal derived from the right eye electrical signal to another of the signal derived from left eye electrical signal or the signal derived from the right eye electrical signal.

10. The system of claim 7, wherein the actions further comprise:
determining a time of relative alignment based on the correspondence; and
identifying the misaligned eye based on a comparison of the signal derived from the left eye electrical signal and the signal derived from the right eye electrical signal at the time of relative alignment.

11. A method of determining an alignment status of a patient's eyes, the method comprising:
directing polarized light to a left retina of the patient and directing polarized light to a right retina of the patient;
obtaining a left eye electrical signal corresponding to polarized light reflected from the left retina for circular scans of the left retina and obtaining a right eye electrical signal corresponding to polarized light reflected from the right retina for circular scans of the right retina;
determining a correlation of a signal derived from the left eye electrical signal with a signal derived from the right eye electrical signal;
determining an alignment status of the patient's eyes based on the correlation; and
providing the alignment status.

12. The method of claim 11, wherein the alignment status comprises an indication of one of: the right eye and the left eye are aligned, or the right eye and the left eye are misaligned.

13. The method of claim 11, wherein the signal derived from the left eye electrical signal comprises a fast Fourier transform power of at least one frequency characteristic of central fixation, and wherein the signal derived from the right eye electrical signal comprises a fast Fourier transform power of at least one frequency characteristic of central fixation.

14. The method of claim 11, wherein the correlation comprises a magnitude-squared coherence of the signal derived from the left eye electrical signal and the signal derived from the right eye electrical signal.

15. The method of claim 11, wherein the correlation comprises a linear fit of a one of the signal derived from the left eye electrical signal or the signal derived from the right eye electrical signal to another of the signal derived from left eye electrical signal or the signal derived from the right eye electrical signal.

16. The method of claim 11, wherein the correlation comprises a spectral correlation coefficient for a signal derived from the left eye electrical signal and a signal derived from the right eye electrical signal.

17. The method of claim 11, wherein the alignment status comprises an identification of a misaligned eye.

18. The method of claim 17, further comprising:
determining a time of misalignment based on the correspondence; and
identifying the misaligned eye based on a comparison of the signal derived from the left eye electrical signal and the signal derived from the right eye electrical signal at the time of misalignment.

19. The method of claim 18, further comprising, prior to the identifying the misaligned eye:
linearly fitting of one of the signal derived from left eye electrical signal or the signal derived from the right eye electrical signal to another of the signal derived from left eye electrical signal or the signal derived from the right eye electrical signal.

20. The method of claim 17, further comprising:
determining a time of relative alignment based on the correspondence; and
identifying the misaligned eye based on a comparison of the signal derived from the left eye electrical signal and the signal derived from the right eye electrical signal at the time of relative alignment.
